(19) United States
(12) Patent Application Publication
(10) Pub. No.: US 2004/0081977 A1
(43) Pub. Date: Apr. 29, 2004

(54) Genetic algorithm convergence accelerating apparatus, system and method for the same

(76) Inventors: Hsien-Tsung Hsu, Taipei (TW); Yue-Ru Chuang, Taipei (TW)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/401,391

(22) Filed: Mar. 28, 2003

(30) Foreign Application Priority Data
Oct. 25, 2002 (TW) 91125176

(51) Int. Cl. C12Q 1/68; G06N 3/00; G06N 3/12

(52) U.S. Cl. 435/6; 702/20; 706/13

(57) ABSTRACT

A genetic algorithm convergence accelerating apparatus, system and method for the same are tools and working methods applicable to computer algorithm to rapidly converge an algorithm result that is close to an optimal solution. The accelerating apparatus includes a chromosome generator for generating a plurality of parent chromosomes having data codes different from each other; a chromosome amplifier having at least a crossover component and a plurality of mutation components for reproducing a plurality of offspring chromosomes having data codes different from each other and calculating fitness values of the offspring chromosomes, so as to compare with the parent chromosomes; an offspring candidate pool for collecting the offspring chromosomes that pass the process of comparing the fitness values before being released in batches; and an offspring pool for selecting the offspring chromosomes fitted for the next crossover from each group of offspring candidates, such that the crossover for the next generation can occur with pairs of the offspring chromosomes or through coupling of the offspring chromosomes with the parent chromosome not yet involved in the crossover. With a fast flow from one group to another, each quadrant in the system is made occupied under the same system execution time to shorten the system idol time, whereby the convergent speed is accelerated to meet time requirement for the real-time system of high speed computer. Furthermore, as the hyper-generation crossover generates offspring with a higher fitness value and in a greater number within a unit time, the convergent result is obtained faster and closer to the optimal solution.

### FIELD OF THE INVENTION

The present invention relates to a genetic algorithm convergence accelerating apparatus, system and method for the same, applicable to industry for processing data in huge amount and high complexity with computer, and more particularly, to a genetic algorithm convergence accelerating apparatus, system and method for the same, established by combining a hyper-generation crossover concept and a pipeline method.

### BACKGROUND OF THE INVENTION

An evolution sequence that occurs in nature has created a powerful solution for a complicated problem. For instance, a biological organism needs to adapt well to changes in a living environment in order to survive and reproduce, so as to prevent its race from going extinction. However, the intelligence required for the biological organism to adapt and survive in the environment is passed from parents down to offspring via chromosomes. From a biological point of view, each of the chromosomes is seen as Deoxyribonucleic Acid (DNA) molecules twisted in a super-coiled form. The chromosome begins to work by partially loosening the coiled structure to form two strands of long DNA pairing chains. The parental genetic information is represented by arrangement and structure of the DNA, which information is replicated from the parents to the offspring through complex heredity, so that the offspring is inherited with characteristics and features (such as skin color, races and so on) similar to the parents.

According to Darwin's theory of evolution, a species that is fittest to survive is produced through a continued evolution of the species. A Genetic Algorithm (GA) proposed by John Holland in 1975 starts out from such concept, which algorithm simulates genetic evolution in nature to search for a manual computational tool for solving many difficult problems in the computer. It has been shown in many experiments that the genetic algorithm is a method for searching an optimal solution with both effectiveness and effect. And the genetic algorithm is also widely applicable to problems related to calculation and setting of a huge amount of data and parameters, data processing sequence, timing closure, packet scheduling, and real-time system in a calculator.

Just like the biological organism, which locates a large group of genes (the smallest basic heredity unit) in the chromosome in a specific way, each possible solution to the problem in the genetic algorithm can be seen as a linear chromosome (known as a chromosome in brief herein after) made of a string of problem parameters. Each chromosome is digitalized by a binary encoding method, such that each chromosome has its own data code for detecting expression of the chromosome in the simulated environment. Through crossover, mutation, and selection of the chromosomes generation by generation, the optimal solution is converged by generating better quality offspring and selecting out poor quality chromosome member continuously (D. E. Goldberg, “Genetic Algorithms In Search, Optimization, and Machine Learning”, Addison Wesley (1989)).

By an in-depth study of an algorithm mechanism, it was understood that the genetic algorithm simulates the biological evolution and transforms the species behaviors such as crossover, mutation, and selection in the natural selection environment into operators, such as crossover component, mutation component, and selection component, responsible for executing a variety of computation functions in the simulated environment. And to make disclosure of the invention more clear, definition for each operator component is described herein. The term “crossover” means genes from paternal and maternal sides are recombined and inherited to the offspring, whereas the term “mutation” means when the parental genes are inherited to the offspring, they are influenced by environment or other factors to produce variations, so that the offspring has a different genotype from the parents. As a result, the direction of offspring evolution is more versatile and the offspring genes are not limited to only a few arrangement combinations. For the term selection, it means arranging the fitness order of the chromosomes based on the fitness value of the offspring chromosomes. The fitness value, in this case, means the level closest to the optimal solution designed according to the problem by the program designer. Therefore, the fitness value can guide the direction of evolution towards the direction requested by the designer, without making the evolution going aimlessly. So, a higher fitness value indicates that the offspring chromosome may be the optimal solution with higher possibility. Accordingly, the offspring chromosome with a higher fitness value may be selected as the parent chromosomes for the next crossover more easily.

To further understand the operation process for the conventional genetic algorithm, a description is made with chromosomes P and Q as examples in the successive evolution. As shown in FIGS. 6A through to 6D, the chromosome (i.e. possible solution) to be involved in the optimal search is encoded by the binary encoding method. Two chromosomes P and Q (i.e. two possible solutions) are randomly selected for crossover in different methods to produce a plurality of new offspring chromosome pairs (P′, Q′) and (P″, Q″). For example, in one generation, there will be one offspring chromosome P that is different from the parent chromosomes out of every one hundred offspring generated, resulting a mutation frequency of 1%. While the mutation frequency is reached, the mutated offspring is expected to create a superior offspring chromosome R (2, 7, 3). After all the offspring is mutated sequentially according to the mutation frequency, each offspring is sent to the selection component for selection so as to keep the chromosome with high fitness value to continue the evolution and to gradually converge to an optimization result. The optimization is a process of searching the most appropriate solution to the problem, given that there are many limitations and conflicting factors. So even if the absolute optimal solution to the problems (such as many undefined problems in the computer calculation) is not obtained, the optimization result is still the most appropriate balanced point to resolve many conflicting factors. Based on such optimization result, the computer parameters are set to provide significant improving effect for the problems (such as searching an optimal setup for the real-time system of sketching software so as to obtain a satisfied diagram production speed).

However, with the conventional genetic algorithm described above, every chromosome members in the same offspring group need to be calculated in sequence. So, acquisition of the optimization result via convergence of offspring chromosomes with high fitness values usually requires many generations of evolution, causing the evolution speed (i.e. time from the start of evolution to the acquisition of optimal solution, also known as convergent speed) to slow down. As a result, more time is wasted on computer calculation.

To improve from the problems above, the program developer proposed a steady-state genetic algorithm to compensate for the slow convergent speed.

As shown in FIG. 7, the steady-state genetic algorithm involves selection of two parent chromosomes with highest fitness values from the parent groups for executing crossover or replication operation. One neo offspring chromosome is selected from the offspring chromosome generated via the crossover path and the offspring chromosome generated via the replication and mutation paths, such that the neo offspring chromosome shall not be the same as the parent chromosome or chromosomes from any other parent groups. Then, the member with the worst fitness value in the parent group is substituted with the neo offspring chromosome to re-establish a new parent chromosome group. The steady-state genetic algorithm achieves the accelerated convergence by following principles such as “reduction in calculation times” and “maintenance of no repeat code among chromosomes”. However, the steady-state genetic algorithm selectively adopts a few amounts of genes for crossover and mutation, lacking the evolution basis of “selection from a large group for replication of superior offspring”. For this reason, the benefit of optimized offspring evolution is not fully utilized, and improvement of the optimal convergent speed is affected. Moreover, the system usually needs to run a huge amount of code matching and mutation actions in order to maintain individuality of genetic code among neo offspring in the evolution process, resulting a prolong evolution time for each generation. Thus, the optimal convergent speed is not improved significantly, nor is time requirement for real-time system of the high performance computer is satisfied.

Bearing the above problems in mind, manufacturers and program developers are eagerly searching solutions to issues, such as accelerating the convergent speed to avoid a very long computation time, and even to meet the time requirement of the real-time system of optic cable network.

### SUMMARY OF THE INVENTION

In light of the problems associated with the conventional art, an objective of the present invention is to provide a genetic algorithm convergence accelerating apparatus, system and method for the same to accelerate genetic algorithm convergent time and to reduce time spent on searching the optimal solution to problems, such as packet scheduling and channel allocation, so as to satisfy the time efficiency requirement of real-time system of the computer.

Another objective of the present invention is to provide a genetic algorithm convergence accelerating apparatus, system and method for the same, which apparatus, system and method have advantages of conventional genetic algorithm and steady state genetic algorithm, avoid long calculation time from one generation to another, and obtain a higher number of offspring chromosomes with higher fitness values than the typical genetic algorithm does under the same time lag, so as to improve the ability to converge the optimal solution rapidly.

As embodied and broadly described herein, the invention provides a hyper-generation genetic algorithm convergence accelerating apparatus, system and method for the same, established based on a pipeline method. The genetic algorithm convergence accelerating apparatus comprises of a chromosome-generating module for generating a plurality of parent chromosomes, with each parent chromosome having a data code different from others, and a chromosome amplifying module group for reproducing offspring chromosomes and deducing a functional equation (F) for calculating fitness value according to a path to search the optimal solution. The chromosome amplifying module group has at least a crossover module and a plurality of mutation modules, wherein the crossover module receives parent chromosomes from the first generation or parent chromosomes from later generations belonging to different groups for crossover, so as to generate offspring chromosomes, and the mutation module serves to increase diversity among offspring generations, such that the neo offspring chromosomes have encoding features different from other offspring chromosomes. The genetic algorithm convergence accelerating apparatus further comprises of a filter for storing the parent and offspring chromosomes before and after the crossover and selecting a plurality of chromosomes with higher fitness values by comparison of their fitness value, so as to ensure a continued upgrade in the fitness value; an offspring candidate database for storing the offspring chromosomes generated from the filter, wherein the offspring chromosomes (also known as offspring candidates) are classified on a time quadrant into a plurality of batches to be released and sent to a selection module; and an offspring database connected to at least the selection module, which selection module selects and replicates the offspring chromosomes fitted for the next crossover from each batch of the offspring candidates before the offspring chromosomes are stored in the offspring database. Once the selected offspring chromosomes appear in the offspring database, the offspring chromosomes thereof are sent into the chromosome amplifying module group for the next crossover.

In addition, from the hardware point of view, the present invention also comprises of a genetic algorithm accelerating apparatus to enhance the processing unit for conducting a data optimization computation function. The accelerating apparatus comprises of a chromosome generator for generating a plurality of parent chromosomes having data codes different from each other and sending the parent chromosome that arrives early to a latch via a multiplexing device and a demultiplexing device, so as to wait for another parent chromosome that arrives late in order to achieve a synchronized crossover of the parent chromosomes executed by the crossover component; a chromosome amplifier having at least a crossover component and a plurality of mutation components formed therein, with the crossover component receives the parent chromosomes sent from the demultiplexer to generate offspring via the crossover, and the mutation components increase diversity among offspring generations and versatility of direction for the offspring evolution, so as to avoid limiting offspring genes to only a few arrangement combinations; an offspring filter for calculating and comparing fitness values for the parent and offspring chromosomes, so that a plurality of chromosomes with higher fitness values are selected as offspring candidates; an offspring candidate pool for collecting the chromosomes selected by the offspring filter as the offspring candidates, wherein the offspring candidates are released in batches on the time quadrant; and an offspring pool connected to at least a selecting component, for selecting, replicating, and storing the offspring chromosomes fitted for the next crossover, such that the fitted offspring chromosome can meet with another parent or offspring chromosome not yet involved in the crossover to repeat crossover operation for the chromosomes of the next generation.

As the genetic algorithm convergence accelerating apparatus is applied to the multi-channel optic cable network to solve complex problems such as optimization of packet scheduling and channel allocation, the chromosome to be involved in the optimization are encoded first to achieve hyper-generation crossover for the parent chromosome group. The optimization solution is searched by the genetic algorithm convergence accelerating method using “group” as a unit to substitute the reproduction of chromosome in batch generation by generation in the conventional method. The method comprises the following steps. First of all, the chromosome-generating module is commanded to generate a plurality of parent chromosomes having data codes different from each other. Then, the chromosome amplifying module group is commanded to select two pairs of parent chromosomes for crossover in order to reproduce the offspring chromosomes, and the offspring chromosomes are subject to mutation, so that each offspring chromosome has an encoding feature different from other offspring chromosomes. Next, the chromosome amplifying module group is commanded to calculate a fitness value for each of the offspring chromosomes and to compare the fitness value of the offspring chromosome with that of the parent chromosome (that is, the filtering process), so as to select two offspring chromosomes with higher fitness values as offspring candidates. Further, the offspring candidate database is commanded to store the offspring candidates, which are classified according to the time quadrant into a plurality of batches (such as nth group, n+1th group, and so on) to be released. And the offspring chromosomes fitted for next crossover is selected and replicated from each batch of the offspring candidates by the selecting module before the fitted chromosomes are stored in the offspring database. And while the previous group of offspring candidates (such as nth group offspring candidates) are sent in the offspring database for selection and replication, the next group of offspring candidates (such as the n+1th group offspring candidates) accumulate immediately and enter the offspring candidate database, such that the chromosomes from the previous and next groups are separated by only one operation step.

According to the present invention, the genetic algorithm accelerating convergence apparatus, system and method for the same make selection of the offspring chromosomes with high fitness values based on the group. The apparatus, system and method also adopt the pipeline concept of synchronized execution in batch where the n+1th group offspring candidates are generated immediately to continue the selection operation after the nth group offspring candidates completed with selection operation enter the next step. So when the first group offspring chromosomes are generated, a part of the parent chromosomes not yet involved in the crossover operation may execute crossover operation with the first group offspring chromosomes by a concept known as hyper-generation crossover (that is, the parent and offspring chromosomes can execute crossover operation). When the whole system is operated, the speed of clearing out the offspring chromosomes in the offspring database will be lower than the speed of offspring chromosomes going into storage in the offspring database. Therefore, the size of the “group” in the offspring candidate database will gradually increase (that is the number of chromosome in the n+1th group will be twice of that in the nth group) until all parts of the parent chromosomes complete the crossover operation. At this time, the parent chromosomes required by the crossover component to execute crossover will come totally from the offspring chromosomes in the offspring database. So, the speed of clearing out the offspring chromosomes in the offspring database will be equal to the speed of offspring chromosomes going into storage in the offspring database. As a result, the system operation enters a saturated state, with the size of the group increases no more once a saturated value is reached. Accordingly, with the hyper-generation crossover concept and a fast flow from one group to another, each quadrant in the system is made occupied under the same system execution time to shorten the system idling time, whereby the convergent speed is accelerated to meet the time requirement for the real-time system of the high speed computer, and the convergent result is closer to the optimal solution.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a schematic diagram illustrating a hardware layout of a genetic algorithm convergence accelerating apparatus according to the present invention;

FIG. 2 is a block diagram illustrating a genetic algorithm convergence accelerating method according to the present invention;

FIG. 3 is a flow chart illustrating the genetic algorithm convergence accelerating method according to the present invention;

FIG. 4 is a schematic diagram illustrating a comparison of neo offspring chromosome amount produced by the conventional genetic algorithm, the steady-state genetic algorithm, and the hyper-generation genetic algorithm according to the present invention within the same period;

FIG. 5 is a schematic diagram illustrating a comparison of optimization speed of packet scheduling and channel allocation when the conventional genetic algorithm, the steady-state genetic algorithm, and the hyper-generation genetic algorithm according to the present invention are applied to the optical fiber network;

FIG. 6A is a schematic diagram illustrating the parent chromosome encoded by the binary encoding method in the conventional genetic algorithm;

FIG. 6B is a schematic diagram illustrating a crossover operation in the conventional genetic algorithm;

FIG. 6C is a schematic diagram illustrating a mutation operation in the conventional genetic algorithm;

FIG. 6D is a schematic diagram illustrating a selection operation in the conventional genetic algorithm; and

FIG. 7 is a flow chart illustrating conventional steady genetic algorithm in operation.

### DETAILED DESCRIPTION OF THE PREFERRED EMBODIMENTS

A genetic algorithm accelerating convergence apparatus and method for the same is an optimization tool for accelerating computer operation, applicable to a central processing unit (CPU) for executing calculation and setting of a huge amount of data and parameters, data process ordering, timing closure, packet scheduling, and real-time system. The tool comprises of a hardware layout and a software operation. So, the actual operations involved in the present embodiment are described with respect to the software and hardware as below.

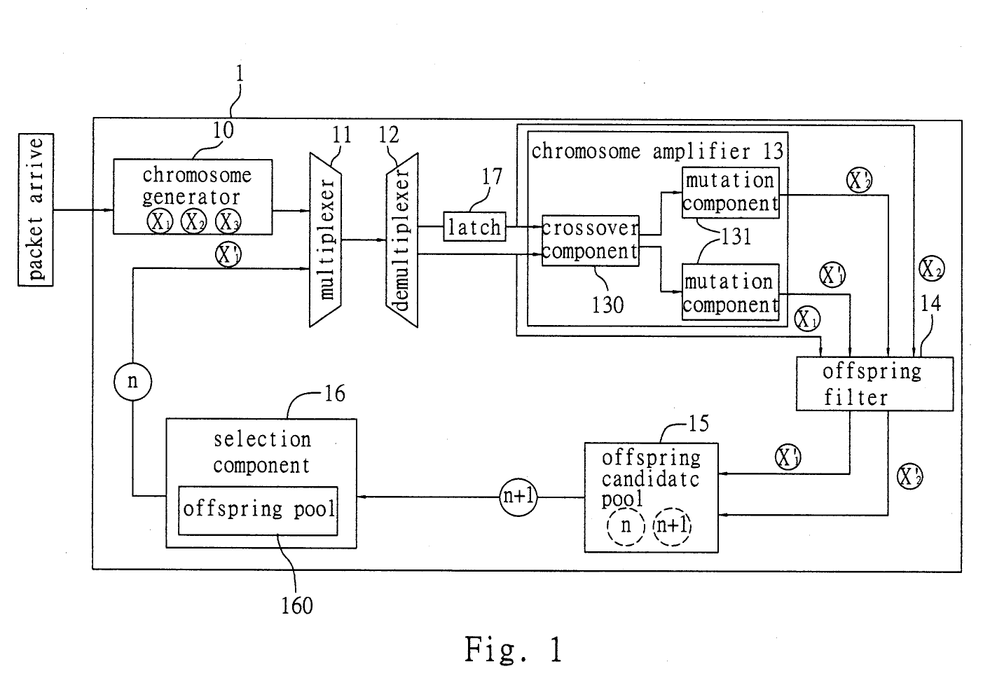

The description begins from the hardware layout. The genetic algorithm accelerating convergence apparatus is a system tool that assists a user who faces with a huge amount of data and highly complex operation, to accelerate a search for an optimization solution. As shown in FIG. 1, the genetic algorithm accelerating convergence apparatus 1 comprises a chromosome generator 10 for generating a plurality of parent chromosomes having different data codes, wherein the parent chromosomes form a parent chromosome group (for example, a first generation chromosome group is constituted by 100 parent chromosomes). A parent chromosome X1 is then selected from the parent chromosome group, which parent chromosome X1 is transmitted via a multiplexing device 11 and a demultiplexing device 12 to a latch 17 for temporary storage. After another parent chromosome X2 is produced in the chromosome generator 10, both chromosomes X1 and X2 are subjected to crossover. And the genetic algorithm accelerating convergence apparatus 1 comprises of a chromosome amplifier 13 having at least a crossover component 130 and a plurality of mutation components 131, so that the parent chromosomes X1 and X2 can crossover, mutate and produce offspring chromosomes X1′, X1″, X2′, and X2″. However, execution of mutation depends on a mutational frequency. That is, whether X1′ and X2′ become X1″ and X2″ or not depends on mutational frequency of each chromosome. The genetic algorithm accelerating convergence apparatus 1 further comprises an offspring filter 14, for calculating and comparing fitness values for each parent chromosomes X1 and X2 and its offspring chromosomes X1″(or X1′) and X2″(or X2′), and an offspring candidate pool 15, for storing two offspring chromosomes (such as X1″ (or X1′) and X2″ (or X2′)) selected from the offspring filter 14 as offspring candidates. So, the offspring chromosomes (such as X1″ (or X1′) and X2″ (or X2′)) with higher fitness value are selected in the offspring filter 14 and sent into the offspring candidate pool 15. The genetic algorithm accelerating convergence apparatus 1 also comprises an offspring pool 160 connected to at least a selection component 16, for selecting and replicating an offspring chromosome fitted for next crossover from the offspring candidates (such as X2″(or X2′)), while the offspring candidate is sent to the latch 17 until another parent chromosome (such as X3) appears. Then, the offspring chromosome can repeat the crossover operation with the parent chromosome.

The genetic algorithm convergence accelerating apparatus with its components and their roles are described with reference to FIG. 1 below, and with packet scheduling as an example for illustrating operation process of the genetic algorithm accelerating apparatus.

As shown in FIG. 1, the chromosome generator 10 generates a plurality of parent chromosomes (such as X1, X2), with each chromosome representing a string of scheduled packet encoded with a binary code. For example, the chromosome P has a code of 0100011. Meanwhile, each chromosome in the same group (a group means an accumulation of chromosome group or generation) is encoded with a different data code. And it should be noted that the present invention is not limited to only the binary encoding method, decimal, sexidecimal, and other encoding methods are also applicable to the present embodiment.

The parent chromosome X1 is transmitted via the multiplexer 11 and the demultiplexer 12 to the latch 17 until the parent chromosome X2 is produced in the chromosome generator 10. Then chromosomes X1 and X2 are synchronously sent in the chromosome amplifier 13 for reproducing offspring. The chromosome amplifier 13 has at least a crossover component 130 and a plurality of mutation components 131. After the parent chromosomes X1 and X2 enter the crossover component 130, the offspring chromosomes X1′, X2′ or X1″, X2″ are produced by methods such as one-point crossover, two-point crossover, or uniform crossover. And if the mutational, frequency permits (for example, if there is one mutated offspring chromosome out of every one hundred offspring chromosome reproduced by a chromosome in Y generation), the mutation operation can be executed using the mutation component 131. Further, the fitness value of each parent chromosome and offspring chromosome is calculated and compared by the offspring filter 14, such that the chromosome with the higher fitness value is kept in the offspring candidate pool 15.

Since the chromosome generator 10, chromosome amplifier 13, crossover component 130, mutation component 131, and the offspring filter 14 described above are well known to those skilled in the art, the details thereof are omitted herein. The present invention is different from the conventional art in that an offspring candidate pool 15 and an offspring pool are formed in the genetic algorithm convergence accelerating apparatus 1 to provide a grouping concept and a pipeline technology, so as to accelerate the execution of convergence operation significantly.

The offspring candidate pool 15 collects the chromosomes that are filtered by the offspring filter 14. And based on a batch concept, the offspring candidates are released by group in a time sequence. As described in the present embodiment, the offspring chromosomes and the parent chromosomes are ordered according to the fitness values, so that the chromosomes (such as X1′ and X2′) with the higher fitness values are selected as offspring candidates, while those chromosomes that do not pass the selection are discarded. After the offspring candidate that satisfy the fitness value enters the offspring candidate pool in sequence, the plurality of offspring chromosomes are further classified into a plurality of groups (such as nth and n+1th groups in FIG. 1), so as to be released in groups by a first in first out method. The group classification depends on the time the offspring candidate is sent into the selection component 16 for selection. For example, when the offspring candidates of the nth group are sent into selection component 16 for selection in FIG. 1, the offspring candidates that come after are classified as n+1th group.

After the selection component 16 receives the offspring candidate of the nth group, the offspring chromosomes fitted for the next crossover are selected based on the fitness values of the offspring candidates in the group and replicated before saving in the offspring pool 160. A ratio calculated from the fitness value of each offspring candidate out of the total fitness values of the offspring candidates in the group may be used as a basis for selection and replication in a method known as roulette wheel method. When the qualified offspring chromosome (such as X1′) appears in the offspring pool 160, the offspring chromosome X1′ is sent to the gate immediately to meet with another parent chromosome X3, so as to execute next crossover operation.

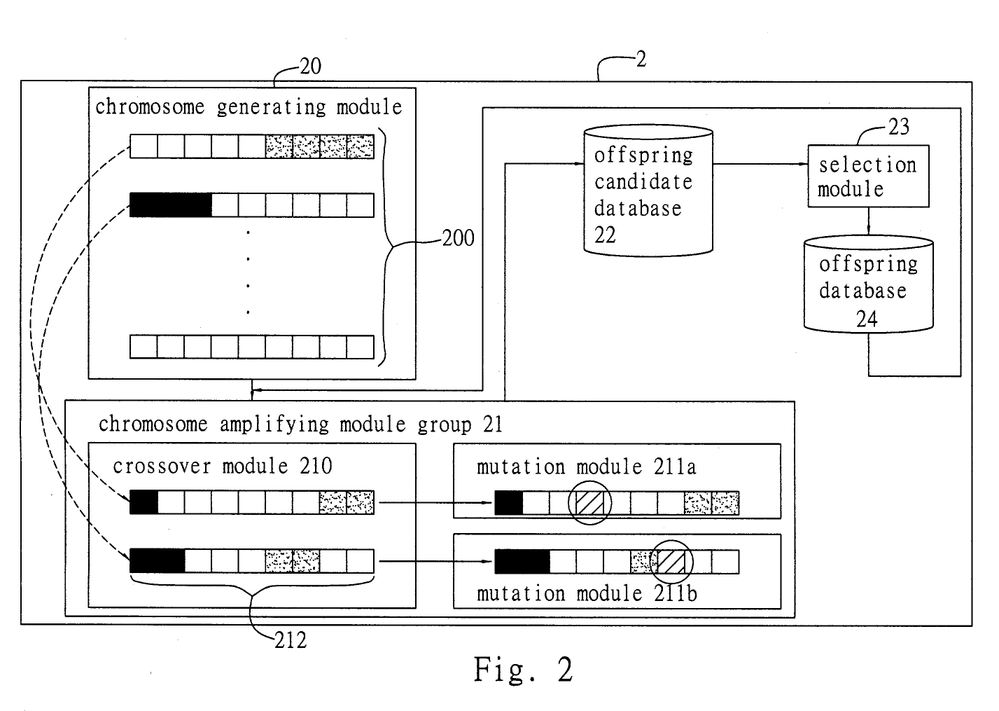

From the software point of view, in order to obtain the optimization solution to problems, such as packet scheduling and channel allocation of high speed computer network, the present invention is also seen as a hyper-generation genetic algorithm convergence accelerating system and method for the same, with the pipeline method as a basis. As shown in FIG. 2, the genetic algorithm convergence accelerating system 2 comprises a chromosome generating module 20, for receiving and transforming external data into a plurality of parent chromosomes 200 with different data codes; a chromosome amplifying module group 21, having at least a crossover component 210 and a plurality of mutation components 211a, 211b for generating offspring chromosomes 212 with different encoding features; an offspring candidate database 22, for saving the offspring candidate chromosomes 212, so that the selected offspring candidates 212 are classified into a plurality of groups according to a time quadrant and released by First In First Out (FIFO) method; and an offspring database 24 connected to at least a selection module 23 for selecting, replicating, and saving the offspring chromosomes (not shown) fitted for the next crossover, such that the crossover operation can be continued for the next generation after the chromosome amplifying module group 21 receives the qualified offspring.

The chromosome generating module 20 receives the external data that passes information exchange, so that the solution forms a plurality of parent chromosomes (not shown). Then, the parent chromosomes are transformed into binary codes by an encoding method or displayed in other encoding formats, wherein each parent chromosomes 200 carries a data code different from other chromosomes 200 in the same generation. Thus, the data code has its own individuality.

The chromosome amplifying module group 21 serves to reproduce offspring chromosomes 212. The chromosome amplifying module group 21 comprises of at least a crossover component 210 and a plurality of mutation components 211a, 211b, wherein the crossover component 210 receives the parent chromosomes for crossover so as to produce offspring chromosomes 212, and the mutation components 211a, 211b serve to increase diversity between the offspring and increase multiple evolution directions for the offspring chromosome 212, so as to avoid limiting the offspring genes to a few arrangement combinations. In addition, the chromosome amplifying module group 21 may also calculate the fitness value for each offspring chromosome. And since functions of the crossover component 210, and mutation components 211a, 211b, as well as calculation of the fitness value have been clearly discussed in the above description for hardware layout, the details thereof are omitted herein.

The offspring candidate database 22 stores the chromosomes (also known as offspring candidates) (not shown) that pass the filter module, and classifies the chromosomes into groups according to order for the offspring candidates to enter the database. So, offspring chromosomes from the nth group, n+1th group, and n+2th group (not shown) all follow the first in first out concept and are released group by group. However, as storage and release executed by each offspring candidates in the database is determined using the data code, each chromosome is taken as a number data. And offspring candidate database in the present embodiment is also applicable to any database that provides data storage or classification.

The offspring database 24 selects, replicates, and stores the offspring chromosome fitted for the next crossover from each group of offspring candidates. A selection and replication may be made based on comparison of fitness value of each offspring candidate to the total fitness value of the offspring candidates in the group in a method, known as roulette wheel method. When a group of the offspring candidates (not shown) are sent into the selection module 23 for selection and replication, the offspring candidates that come after form a new group immediately in the offspring candidate database. Thus, the offspring candidates from the previous group and the next group flow rapidly and smoothly by a pipeline method. And if the flow rate between the groups is fast enough, a hyper-generation crossover occurs between the neo offspring chromosome and the parent chromosome not yet involved in the crossover from the first generation. With such hyper-generation crossover, the rate of chromosome evolution and the group number generated are increased far more than by the conventional genetic algorithm in the same time period. As a result, a larger sample number is obtained, making the converged result to better approximate the optimal solution with the same time requirement.

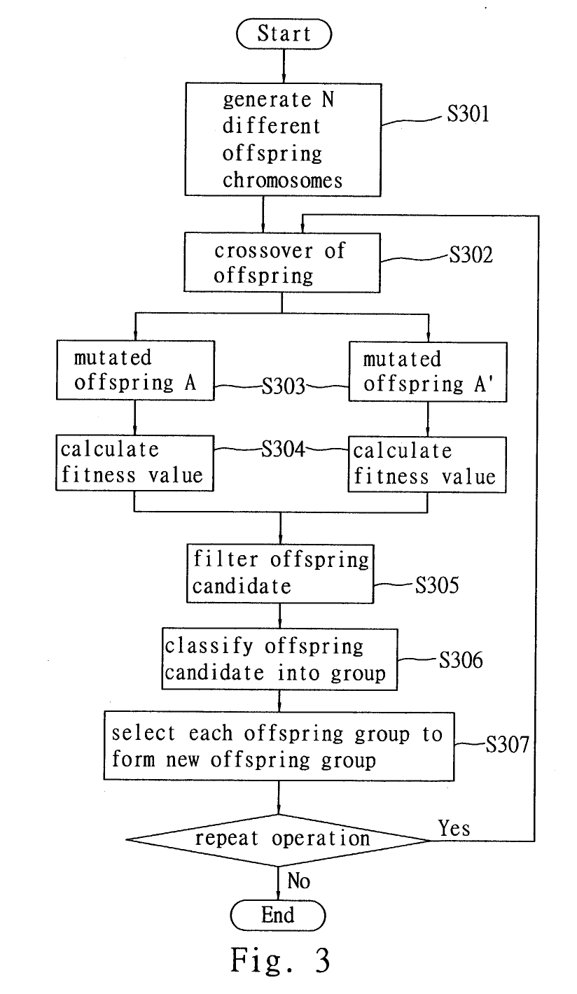

The above system operated by the pipeline genetic algorithm with each group as a unit substitutes a generation-by-generation batch reproduction in the conventional algorithm, so that the chromosomes that represent problem-related variables may be converged rapidly towards an evolution direction with the high fitness value via a fast reproduction of offspring group. As illustrated in both FIGS. 2 and 3, the pipeline genetic algorithm of the present invention comprises the following process steps.

First of all, in step S301, the chromosome generating module 20 is commanded to generate a plurality of parent chromosomes 200, each having a data code different from others. Then, the process moves on to step S302.

In step S302, the chromosome amplifying module group 21 is commanded to receive in order two pairs of parent chromosomes 200 (to form alleles for replication), so as to crossover and reproduce the offspring chromosomes 212. Then, the process moves on to step S303.

In step S303, the mutation modules 211a, 211b are commanded to execute mutation operations, so that each offspring chromosome 212 has an encoding feature different from others. Then, the process moves on to step S304.

In step S304, the chromosome amplifying module group 21 is commanded to calculate fitness value for each offspring chromosome according to the preset function. Then, the process moves on to step S305.

In step S305, the chromosome amplifying module group 21 is commanded to filter and select from the parent chromosomes and their offspring chromosomes for a plurality of offspring candidates with higher fitness values. Then, the process moves on to step S306.

In step S306, the offspring candidate database is commanded to store the offspring candidates, and classify the offspring candidates into a plurality of groups (such the nth group, n+1th group, and so on) by a time quadrant and released group by group. Then, the process moves on to step S307.

In step S307, the selection module is commanded to select the offspring chromosome fitted for the next crossover from each group of the offspring candidate chromosomes. As the selection from the previous group of offspring candidates is complete and the qualified offspring chromosomes are replicated and sent into the offspring database for storage, the next batch of offspring candidates (that is, the next group of offspring candidates) are immediately subjected to selection and replication. With a fast flow from one group to another, the hyper-generation crossover occurs between the neo offspring chromosomes and the parent chromosomes not yet involved in the crossover from the first generation. Once all of the parent chromosomes are done in the crossover, the system immediately enters a saturated closure status, with the chromosome supply for the crossover comes totally from the offspring database.

The pipeline hyper-generation genetic algorithm combines the evolution rule where a superior individual is selected and replicated from a large sample space in the conventional genetic algorithm and the concept of reducing calculation time between generations in the steady state genetic algorithm. So, as the real-time system is taken into account, the hyper-generation genetic algorithm can produce more and better chromosomes than the conventional genetic algorithm and the steady state genetic algorithm under the same time lag, resulting a faster convergent result that is close to the optimal solution.

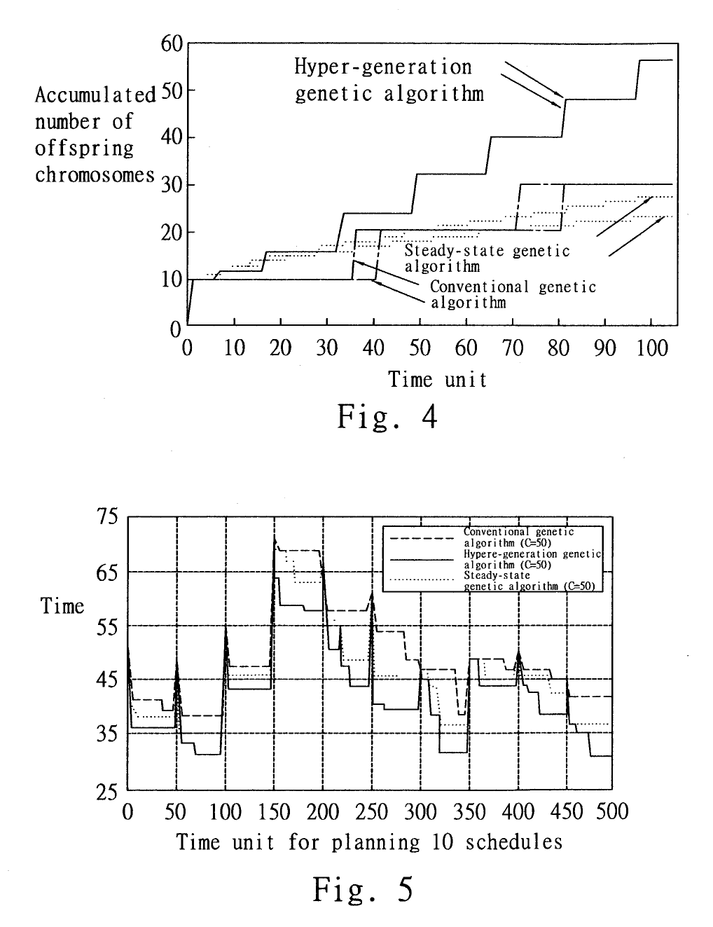

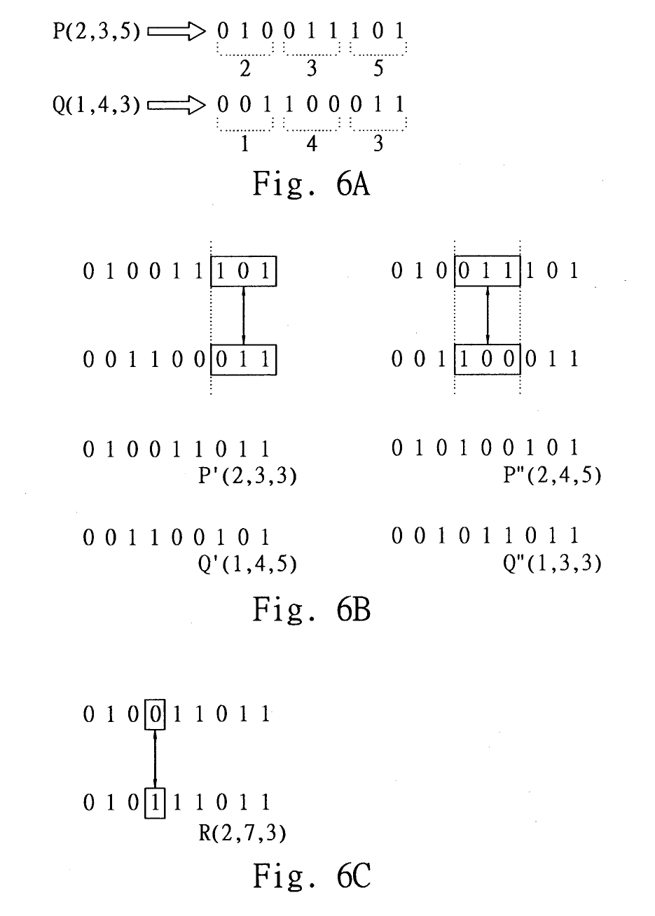

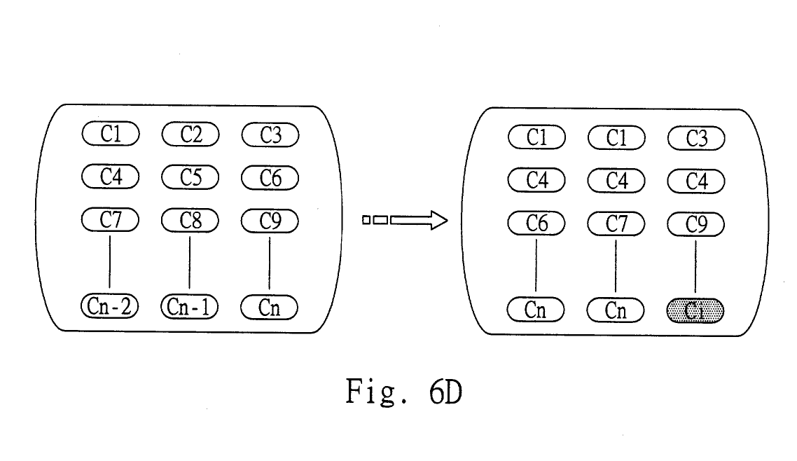

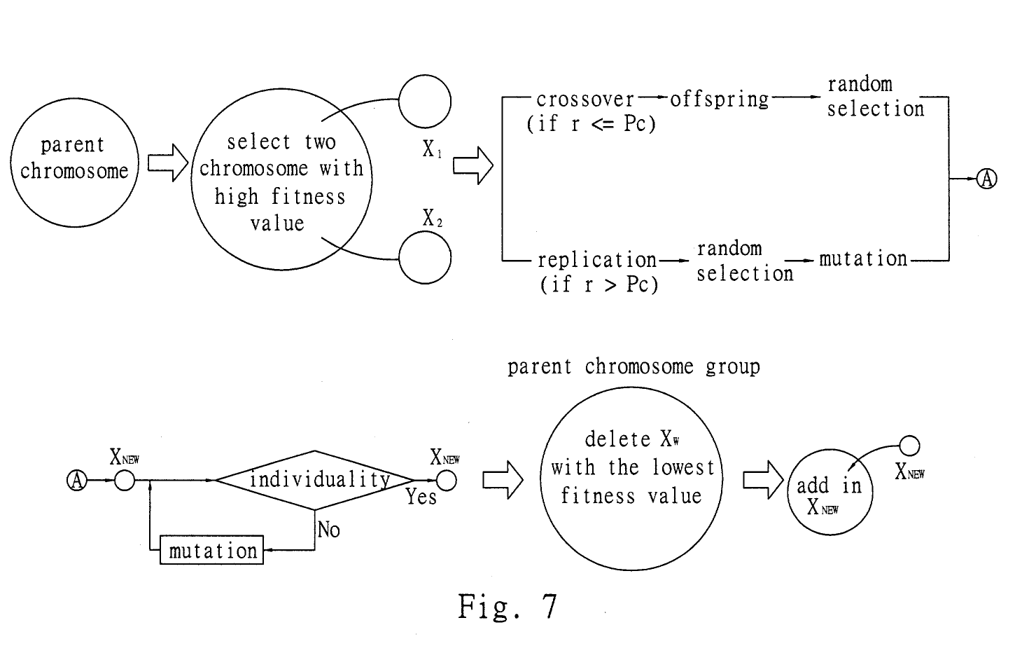

On the other hand, if optimization of packet scheduling and channel allocation for the multi-channel optic cable network is taken as an example of difficult problem, comparing function of the conventional genetic algorithm and steady state genetic algorithm with the hyper generation genetic algorithm of the present invention gives a better picture about the efficiency of applying the hyper generation genetic algorithm to the real time system. As shown in FIGS. 4 and 5, the conventional genetic algorithm, steady state genetic algorithm, and the hyper generation genetic algorithm are simulated respectively as Genetic Algorithm Packet Schedulers (LAGS) to operate the real-time system. From the simulated results, it can be understood that the number of offspring chromosomes generated by the hyper generation genetic algorithm is indeed higher than those generated by the conventional genetic algorithm and steady state genetic algorithm. Meanwhile, the hyper generation genetic algorithm also yields a better scheduling result faster than the conventional genetic algorithm does within a limited time quadrant. Moreover, the hyper generation genetic algorithm can still yield a convergent result closer to the optimization solution even under a more stringent time period.

The invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

What is claimed is:

1. A genetic algorithm convergence accelerating apparatus, applicable to an algorithm for solving computer problems unable to be solved by common computations, so as to rapidly converge an algorithm result that is close to an optimal solution, the accelerating apparatus comprising:
a chromosome generator for generating a plurality of parent chromosomes;
a chromosome amplifier having a plurality of operating components for reproducing offspring chromosomes from the parent chromosomes, such that the offspring chromosomes are in a greater number than the parent chromosomes;
an offspring filter for calculating and comparing fitness values for the parent and offspring chromosomes;
an offspring candidate pool for collecting the chromosomes that pass comparing step in the offspring filter as offspring candidates, wherein the offspring candidates are released in batches; and
an offspring pool connected to at least a selection component, for selecting, replicating, and storing the offspring chromosomes, such that the offspring chromosomes can be coupled to the chromosome amplifier for reproducing offspring chromosomes in next generation.

2. The apparatus of claim 1, wherein the algorithm includes a Hyper-generation Genetic Algorithm.

3. The apparatus of claim 1, wherein the chromosome includes linear chromosomes assembled by a string of problem parameters.

4. The apparatus of claim 1, wherein the chromosome forms a string of data codes by binary, decimal and sexidecimal encoding methods.

5. The apparatus of claim 1, wherein each chromosome member in the parent chromosomes has a data code different from others.

6. The apparatus of claim 1, further comprising a multiplexing device and a demultiplexing device installed between the chromosome generator and the chromosome amplifier.

7. The apparatus of claim 1, the operating component comprising at least a crossover component and a plurality of mutation components.

8. A genetic algorithm convergence accelerating system, applicable to an algorithm for searching an optimal solution to computer problems unable to be solved by common computations, so as to rapidly converge an algorithm result that is close to the optimal solution, the accelerating apparatus comprising:
a chromosome generating module for generating a plurality of parent chromosomes;
a chromosome amplifying module group having a plurality of operating components for reproducing offspring chromosomes from the parent chromosomes, and calculating a fitness value for each of the offspring chromosomes, so as to compare the fitness value of the offspring chromosome with the fitness value of the parent chromosome;
an offspring candidate database for storing the chromosomes that pass fitness value filtering step as offspring candidates, wherein the offspring candidates are classified in a plurality of batches and released in batches; and
an offspring database connected to at least a selection module, for selecting and replicating the offspring chromosomes fitted for next crossover from each batch of the offspring candidates, so as to conduct a new generation crossover with another batch of the offspring chromosomes.

9. The system of claim 8, wherein the algorithm includes a Hyper-generation Genetic Algorithm.

10. The system of claim 8, wherein the chromosome includes a linear chromosome assembled by a string of problem parameters.

11. The system of claim 8, wherein the chromosome forms a string of data codes by binary, decimal and sexidecimal encoding methods.

12. The system of claim 8, wherein each chromosome member in the parent chromosomes has a data code different from others.

13. The system of claim 8, wherein the operating component comprising at least a crossover component and a plurality of mutation components.

14. The system of claim 8, wherein a flow sequence of the offspring chromosome group depends on a first in first out timing quadrant.

15. A genetic algorithm convergence accelerating method, applicable to an algorithm for searching an optimal solution to a computer problem unable to be solved by a common computation, so as to rapidly converge an algorithm result that is close to the optimal solution, the accelerating method comprising steps:
commanding the chromosome generating module to generate a plurality of parent chromosomes;
commanding the chromosome amplifying module group to select two parent chromosomes for crossover so as to reproduce offspring chromosomes, and calculate a fitness value for each of the offspring chromosomes, so as to compare the fitness value of the offspring chromosome with the fitness value of the parent chromosome;
commanding the offspring candidate database to store the chromosomes that pass fitness value filtering step as offspring candidates, wherein the offspring candidates are classified into a plurality of batches to be released; and
commanding the selection module to select and replicate the offspring chromosomes fitted for next crossover from each batch of the offspring candidates, so as to conduct a new generation crossover with another batch of the offspring chromosomes.

16. The method of claim 15, wherein the algorithm includes a Hyper-generation Genetic Algorithm.

17. The method of claim 15, wherein the chromosome includes a linear chromosome assembled by a string of problem parameters.

18. The method of claim 15, wherein the chromosome forms a string of data codes by binary, decimal and sexidecimal encoding methods.

19. The method of claim 15, wherein the operating component comprising at least a crossover component and a plurality of mutation components.

20. The method of claim 15, wherein a flow sequence of the offspring chromosome group depends on a first in first out timing quadrant.

21. The method of claim 15, wherein the chromosome not yet involved in the crossover includes the parent chromosomes.

22. The method of claim 15, wherein the offspring chromosomes serve as supply for the chromosome not yet involved in the crossover when the parent chromosomes are used up.
